(19) United States
(12) Patent Application Publication
(10) Pub. No.: US 2012/0130171 A1
(43) Pub. Date: May 24, 2012

(54) ENDOSCOPE GUIDANCE BASED ON IMAGE MATCHING

(75) Inventors: Yarom Barak, Tivon (IL); Stuart Wolf, Yokneam (IL)

(73) Assignee: C2CURE INC., Wilmington, DE (US)

(21) Appl. No.: 12/949,387

(22) Filed: Nov. 18, 2010

(51) Int. Cl. A61B 1/00

(52) U.S. Cl. 600/117

(57) ABSTRACT

A method of navigating a cavity of a subject employing images from a previous navigation is provided. In a first endoscopic examination, a set of schemas and at least one bookmark are saved in a data storage device. Each schema includes at least a still image in a cavity and a direction of a distal end. At least one bookmark is defined as at least one schema at a point of interest. In a second endoscopic examination, still images during navigation of an endoscope are compared with still images in the set of schemas to find a match, and use the information derived from the match to determine the location and the orientation of the distal end. An endoscopic system for effecting this method and a non-transitory machine-readable storage medium embodying a program for operating such an endoscopic system are also provided.

### BACKGROUND

The present disclosure relates to a method and system for navigating an endoscope, and particularly to a method for navigating an endoscope based on image matching, a system for effecting the same, and a non-transitory machine-readable data storage device embodying a program for effecting the same.

Endoscopes are employed to look inside a cavity of a subject, which is typically a body of a living organism. Endoscopes are widely used for medical purposes to examine the interior of a hollow organ, i.e., a cavity, of the body. Various types of endoscopes are known in the art for many different types of applications. Typically, an endoscope includes an insertion portion having a distal end capable of articulation. An operation portion at a proximal end of the endoscope is used to grasp the insertion portion, and can include controls for use of the endoscope, such as control knobs for controlling the articulation of the distal end. The distal end can have an imaging device, such as a CCD and optics for directing an image onto the CCD. The endoscope can be used with peripheral devices such as an illumination device, having a light source for directing light to the distal end through fiber optics contained in the insertion portion, a video/image processor, and a display. Alternatively, the endoscope can have a lens system at the distal end that transmits images of an examined area through fiber optics in the insertion portion to an eyepiece and/or image capturing device located at the operation portion.

An endoscope is inserted directly into a cavity of an organ to provide images of selected regions of the cavity. As known in the art, the navigation of the tip of an endoscope is based on the analysis of the images generated in-situ during the process of the navigation. In other words, a practitioner, which may be a physician in the case where a human patient is examined, analyzes the images generated by the endoscope during the navigation, and determines how to proceed with further navigation of the distal end. In other words, navigation of the distal end during an examination employs visual information generated solely during that examination. As such, each navigation event accompanying an endoscopic examination is a separate event unrelated to any previous navigation event accompanying a prior endoscopic examination.

In general, prior art endoscopes lack any navigation system that utilizes navigational data from a prior examination. Thus, an operator of an endoscope is forced to navigate an endoscope de novo to reach a point of interest for examination purposes without any benefit of navigational help from prior endoscopic examinations.

For example, if a physician who removed a polyp during an operational procedure desires to check on the region from which the polyp was removed at a later time, there is no visual indication to mark the location of the region of the polyp removal. Such scenarios occur quite often in procedures employing an endoscope, and particularly in procedures employing a bronchoscope, which is a type of endoscope employed to examine the lower respiratory tract.

Another example in which locating a region of interest is difficult occurs when operation and diagnosis are performed separately. Specifically, a procedure may be performed in a clinic to locate a region of interest, i.e., a region including a polyp. Subsequently, the removal of the polyp may be performed in an operating room. The physician in the operating room must navigate an endoscope to the region including the polyp without the navigational information associated with the initial diagnosis that located the polyp in the clinic.

### BRIEF SUMMARY

In the present disclosure, a method of navigating a cavity of a subject employing images from a previous navigation is provided. In a first endoscopic examination, a set of schemas and at least one bookmark are saved in a data storage device. Each schema includes at least a still image in a cavity and a direction of a distal end. At least one bookmark is defined as at least one schema at a point of interest that requires additional examination in the future. In a second endoscopic examination, still images during navigation of an endoscope are compared with still images in the set of schemas to find a match, and use the information derived from the match to determine the location and the orientation of the distal end and needed adjustment in the orientation of the endoscope and/or distance to travel. A region corresponding to a bookmark can be reached based on comparison of the images generated from the endoscope during the navigation and the set of schemas previously generated and stored even when an original feature defining the location of a point of interest is no longer present. An endoscopic system for effecting this method and a non-transitory machine-readable storage medium embodying a program for operating such an endoscopic system are also provided.

According to an aspect of the present disclosure, a system for endoscopic examination includes an endoscope and a computing means. The computing means is configured to perform the steps of: storing data for a set of schemas, each schema including a first still image of a region in a cavity of a subject generated during a first endoscopic examination and a direction of a distal end of the endoscope or another endoscope at a time of taking the first still image; storing a second still image generated by the endoscope in a second endoscopic examination of the cavity; and finding a matching image among a set of first still images in the set of schemas, wherein the matching image and the second still image depict the same region in the cavity.

According to another aspect of the present disclosure, a method of operating an endoscope includes: generating a set of schemas in a first endoscopic examination of a cavity of a subject, each schema including a first still image of a region in the cavity and a direction of a distal end of an endoscope at a time of taking the first still image; generating a second still image in a second endoscopic examination of the cavity; and finding a matching image among a set of first still images in the set of schemas, wherein the matching image and the second still image depict a same region in the cavity.

According to yet another aspect of the present disclosure, a non-transitory machine-readable data storage device is provided, which embodies a program of machine-readable instructions that can be performed in a computing means. The machine-readable instructions includes steps for: storing data for a set of schemas, each schema including a first still image of a region in a cavity of a subject generated during a first endoscopic examination and a direction of a distal end of the endoscope or another endoscope at a time of taking the first still image; storing a second still image generated by an endoscope in a second endoscopic examination of the cavity; and finding a matching image among a set of first still images in the set of schemas, wherein the matching image and the second still image depict a same region in the cavity.

### BRIEF DESCRIPTION OF THE SEVERAL VIEWS OF THE DRAWINGS

FIG. 1 is a schematic see-through illustration of an exemplary endoscopic examination of a subject according to an embodiment of the present disclosure.

FIG. 2 is a first flow chart illustrating an exemplary sequence of processing steps for a first endoscopic examination according to an embodiment of the present disclosure.

FIG. 3 is a second flow chart illustrating an exemplary sequence of processing steps that can be employed to implement processing step 130 in the first flow chart according to an embodiment of the present disclosure.

FIG. 4 is a second flow chart illustrating an exemplary sequence of processing steps for a second endoscopic examination according to an embodiment of the present disclosure.

FIG. 5A schematically illustrates a first exemplary still image generated during a first endoscopic examination according to an embodiment of the present disclosure.

FIG. 5B schematically illustrates a second exemplary still image generated during a second endoscopic examination according to an embodiment of the present disclosure.

FIG. 5C schematically illustrates an exemplary current image and a cursor pointing along an orientation of the second exemplary still image that corresponds to an orientation of a distal end of an endoscope at the time of generation of the second exemplary still image according to an embodiment of the present disclosure.

FIG. 5D schematically illustrates the exemplary current image after rotating a distal end to match the orientation of the cursor with the orientation of the distal end of the endoscope associated with the matching image according to an embodiment of the present disclosure.

FIG. 6 schematically illustrates an exemplary system configured to perform endoscopic examinations according to an embodiment of the present disclosure.

### DETAILED DESCRIPTION

As stated above, the present disclosure relates to a method for navigating an endoscope based on image matching, a system for effecting the same, and a non-transitory machine-readable data storage device embodying a program for effecting the same, which are now described in detail with accompanying figures. It is noted that like and corresponding elements are referred to by like reference numerals. The drawings are not in scale.

As used herein, an “endoscope” refers to any optical instrument configured to generate an image of a region in a cavity of a subject.

As used herein, a “computing means” refers to any device or any embedded component that is configured to perform logical operations and/or mathematical operations on any form of data provided as an electronic or optical signal.

As used herein, a “schema” refers to a unit of data that includes at least one image generated by an endoscope and optionally including additional information relating to a status of the endoscope at the time of generation of the at least one image. Such additional information may include the direction and/or incline of a distal end of the endoscope and/or any other additional information relating to position and/or spatial orientation of the endoscope.

As used herein, a “point of interest” refers to a region in a cavity that an operator or a machine-executable algorithm identifies as a region for additional attention either in the form of additional observations or an operation thereupon.

As used herein, a “bookmark” refers to a still image within a schema that deemed by an operator or a machine-executable algorithm to include a point of interest.

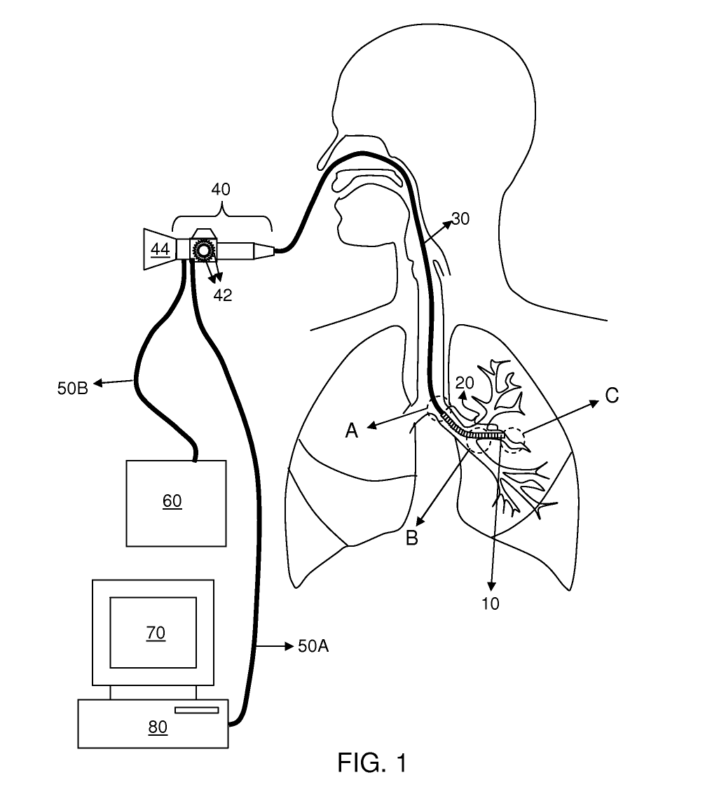

FIG. 1 shows an exemplary endoscopic examination of a subject in a schematic see-through illustration according to an embodiment of the present disclosure. An exemplary endoscope includes a distal end 10, an insertion portion 30 including an articulating section 20, an operating portion 40 that includes control knobs 42 for controlling an articulation of the articulating section 20, an imaging device 44 attached to the operating portion 40, at least one optical fiber bundle 50B connected to a light source 60. A display means 70 and a computing means 80 are electronically connected to the endoscope, for example, by signal cables 50A or by wireless communication. In some embodiments, the display means 70 can be integrated into the operating portion 40 or into the imaging device 44.

A portion of an endoscope including an insertion portion 30, which includes the articulating portion 20, and a distal end 10 is inserted into a cavity of a subject. The subject can be a living organism such as a human or an animal or an inanimate object depending on applications. Any type of endoscope known in the art can be employed provided that the endoscope can be configured to be connected to a computing means for transmission of data and control signals or a computing means is embedded within a portion of the endoscope such as the operating portion 40 of the endoscope. A current image or series of images from the endoscope can be displayed on a display device 70, which can be attached to the computing means 80 or to the operating portion 40 for portability.

The distal end 10 includes device components configured to enable taking a still image, series of images and/or video images. The device components in the distal end 10 may include an imaging device, such as a CCD and appropriate optics for focusing the image onto the imaging device for taking a still image, a series of images and/or video images, and/or may include a lens system configured to transmit optical, infrared, and/or ultraviolet signals to the operating portion 40 located outside the cavity. In case the distal end 10 includes a lens system, a camera or an equivalent device that captures images can be located within the operating portion 40. The endoscope may be configured to transmit the image to a computing means 80 by electronic means through signal cables 50A or wireless communication. In case a camera is included in the distal end 10, the camera may be configured to transmit a still image and/or video images through a set of signal wires in the articlating section 20 and the insertion portion 30 to the operating portion 40 and subsequently to any other device (including at least one computing means 80) configured to process or store such still image and/or such motion picture images. Alternately or additionally, the camera may be configured to transmit a still image and/or video images by wireless communication with any computing means located in the operating portion 40 and/or a separate computing means located outside the endoscope.

The position of the distal end 10 is controlled by a control signal that is provided through signal wires in the insertion portion 30 or communicated through wireless transmission of a control signal from the operating portion 40 or any control means attached, physically or wirelessly, to the operating portion 40. The insertion portion 30 provides mechanical support to the distal end 10 so that the distal end 10 may be navigated through the cavity without being detached or lost.

The articlating section 20 is attached to the distal end 10, and is also referred to as a bending portion. The articulating section 20 can articulate laterally or vertically relative to a lengthwise direction of the articlating section 20 and/or the insertion portion 30 about an articulation joint to allow an articulating end to be angularly oriented relative to an end of the insertion portion 30 that adjoins the articlating section 20.

The insertion portion 30 includes a soft flexible material that contacts the cavity of the subject. The insertion portion 30 can function as a conduit for light from the light source 60 to the distal end, mechanical movement control signals from the operating portion 40 or the computing means 80 to the articlating section 20 and/or the distal end 10, electronic control signals from the operating portion 40 or the computing means to any camera or optics system in the distal end 10, and the electronic signal or the optical signal embodying still images or motion pictures from the distal end 10 to the operating portion 40.

Adjustment of directions and/or orientations of the articlating section 20 and the distal end 10 can be effected by the control knobs 42 attached to the operating portion 40, or can be remotely controlled through the computing means 80. The endoscope may be configured to provide illumination in front of the distal end 10, i.e., in the area of examination, by channeling light from the light source 60 through at least one optical fiber bundle 50B and at least another optical fiber (not shown) in the insertion portion 30 to the distal end 10. The light source 60 may be provided within the operating portion 40, or may be provided as an external component that channels light through the operating portion 40 through at least one optical fiber bundle 50B. The imaging device 44 can be employed to generate still images and/or motion pictures, and can relay the still images or motion pictures to the computing means 80. Alternately or additionally, still images and/or motion pictures can be generated at the distal end 10 and relayed to the computing means 80.

While FIG. 1 illustrates the procedure of bronchoscopy that examines a lower respiratory tract of a human being as an illustrative example of endoscopy, the methods, the systems, and the non-transitory machine-readable data storage devices according to various embodiments of the present disclosure can be employed for any endoscopy procedures known in the art.

Multiple endoscopic examinations can be performed in a cavity of a subject at different times. For example, the multiple endoscopic examinations can include a first endoscopic examination performed in the cavity of the subject at one point in time and a second endoscopic examination performed in the cavity of the subject at another point in time after the first endoscopic examination.

During the first endoscopic examination, at least one point of interest may be identified. Each point of interest can be any region that an operator marks for additional observations or an operation thereupon in the future or a machine-executable algorithm that analyzes images during the first endoscopic examination flags as a region for additional observations or an operation thereupon in the future. In FIG. 1, exemplary points of interest are identified as a first point of interest A, a second point of interest B, and a third point of interest C that the distal end 10 of the endoscope sequentially reaches during the first endoscopic examination. Navigational information is generated and stored during the first endoscopic examination in order to help the navigation of a distal end 10 during the second endoscopic examination. Specifically, information needed to reach the various points of interest (A, B, C) is stored in a data storage device during, or immediately after, the first endoscopic examination, and is subsequently retrieved for comparison with navigational information generated during the second endoscopic examination so that navigation of the distal end 10 to the various points of interest (A, B, C) is facilitated during the second endoscopic examination.

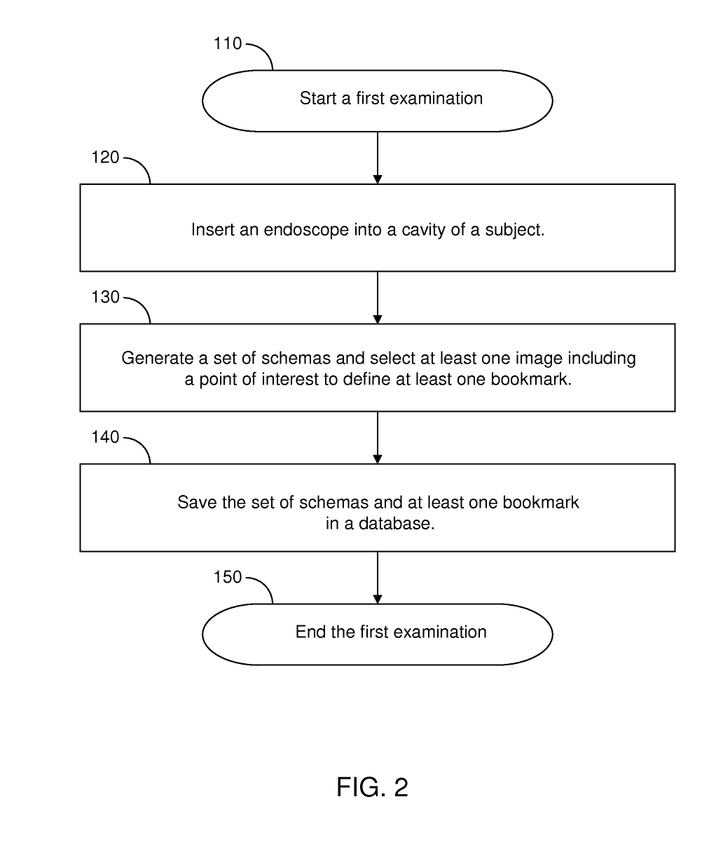

Referring to FIG. 2, a first flow chart illustrates a non-limiting exemplary sequence of processing steps for a first endoscopic examination. Referring to step 110, the first endoscopic examination begins by providing an endoscope and a subject including a cavity to be examined. Control of the first endoscopic examination can be provided by a computing means. Specifically, the endoscope and the computing means can be configured to enable generation of a set of first still images in the first endoscopic examination. The system including the endoscope and the computing means can be configured to generate the set of schemas employing a set of first still images.

Referring to step 120, the endoscope is inserted into the cavity of the subject at an entry point of the cavity.

Referring to step 130, a set of schema is generated and at least one bookmark is defined by selecting at least one image including a point of interest. The set of schema is generated as the distal end moves though the cavity. Each schema corresponds to a position in the path of the distal end at which a first still image of a region of the cavity is taken. Each schema includes a first still image of a region in the cavity and a direction of the distal end of the endoscope at the time of taking the first still image. Each first still image can be taken as a standalone still image or a frame of video images generated at a position of the distal end along the path. The data for the set of schemas is stored in a data storage device.

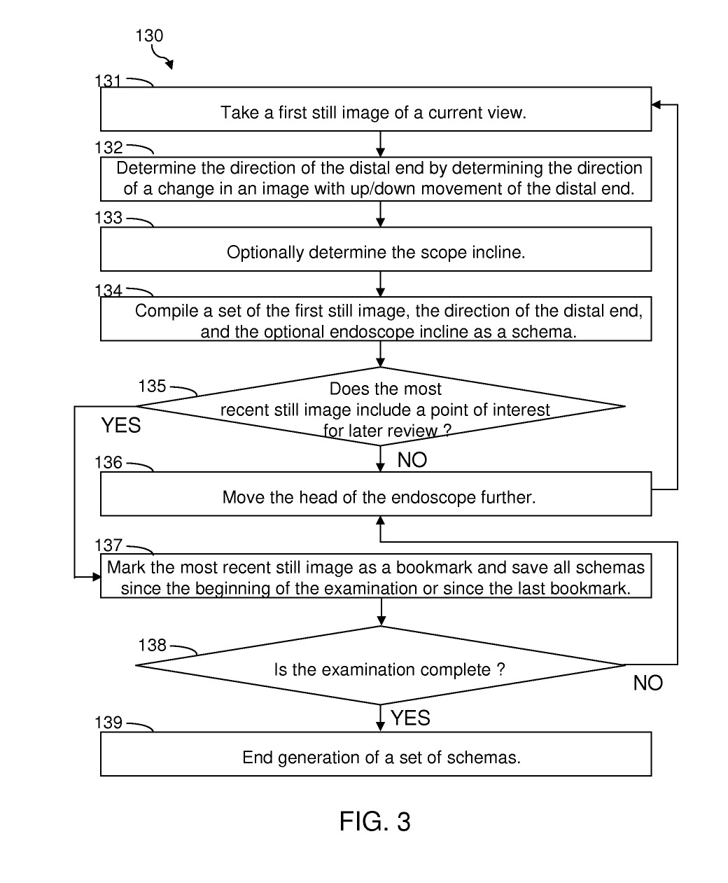

Referring to FIG. 3, a second flow chart illustrates a non-limiting exemplary sequence of processing steps that can be employed to implement processing step 130 in the first flow chart. The processing steps 131-139 can be collectively performed as processing step 130 in the first flow chart of FIG. 2.

Referring to step 131, during the movement of the distal end through the cavity, a first still image of a current view is generated by the endoscope at each moment or location of the distal end as selected according to a predetermined plan. The first still image herein refers to a still image generated during the first endoscopic examination. In one embodiment, first still images can be generated at regular time intervals during the movement of the distal end along the cavity. The regular time intervals can be the same time period employed throughout the first endoscopic examination. The regular time intervals can be predetermined prior to commencement of the first endoscopic examination, or can be determined or changed during the first endoscopic examination. The time period for each regular time interval can be selected from a range from 0.1 second to 20 seconds, and typically from 1 second to 5 seconds, although lesser and greater time periods can also be employed.

In another embodiment, a first still image can be generated at each manually or automatically selected position of the distal end during the first endoscopic examination. Generation of the first still images in this manner can be performed in lieu of, or in addition to, generating first still images at regular time intervals.

Referring to step 132, for each first still image generated by the endoscope during the first endoscopic examination, the direction of the distal end at the time of taking the first still image is determined. The data representing the direction of the endoscope can be determined, for example, by determining the direction of the change in the current image from the endoscope with an up/down movement of the distal end. The up/down movement of the endoscope is a movement of the distal end in a direction perpendicular to an axial direction of an adjoining portion of the articlating section and/or the insertion portion of the endoscope. The up/down movement of the distal end can be automatically actuated with each taking of the first still image. If any first still image is taken upon a manual prompt, the up/down movement of the endoscope can be automatically triggered, or can be triggered upon manual confirmation of the safety or validity of the up/down movement through a human-machine interface (HMI) device such as an up/down actuator in the form of a lever or a button.

Referring to step 133, for each first still image generated by the endoscope during the first endoscopic examination, the scope incline can be optionally determined. Determination of the scope incline can be effected if an inclinometer is attached to the distal end. An inclinometer is a device configured to measure inclination of an object with respect to gravity. The inclinometer incorporated into the distal end can determine the inclination of the distal end, and correspondingly, the spatial orientation of the distal end, at the time of, or about the time of, taking of each first still image.

Referring to step 134, the data representing the first still image, the data representing the direction of the distal end at the time of taking of the first still image, and the optional data representing the inclination of the distal end at, or around, the time of the first still image are complied as a schema. The schema is temporarily saved in a computing means that is configured to receive data from the endoscope. The computing means may be embedded in the endoscope, for example, within the operating portion, or may be a stand-alone computing device such as a personal computer (PC) or a special purpose computer dedicated to controlling and/or communicating the endoscope.

Referring to step 135, determination is made as to whether the most recent still image from the endoscope, i.e., the first still image stored as part of the most recently stored schema, includes a point of interest for later review, either for the purpose of future observations or an operation thereupon. The determination can be made by an operator of the endoscope or by an automatically-executed program that runs on a computing means, which can be the same as, or different from, the computing means in which schemas generated from the first endoscopic examination are stored. If the operator of the endoscope makes a decision, the decision can be based on the operator's analysis of the features in the most recent still image from the endoscope. Alternately, a predefined point of interest may be known to the operator, or may be programmed in a machine-executable program running on the computing means connected to the endoscope. In this case, the determination as to whether the most recent still image from the endoscope includes a point of interest can be made by comparing features in the most recent still image from the endoscope with features expected from the predefined point of interest or the features programmed into the machine-executable program.

If the most recent still image from the endoscope does not include a point of interest, the process flow proceeds to step 136. At step 136, the distal end moves further into the cavity either toward a predetermined destination or in search of any point of interest to be subsequently defined. The process flow proceeds to step 131 again, so that additional first still image is generated upon satisfaction of a condition for taking such additional first still image. The condition for taking such additional first still image can generated by the endoscope at each moment or location selected according to the predetermined plan, e.g., at regular time intervals or at each manually or automatically selected position of the distal end during the first endoscopic examination.

If the most recent still image from the endoscope includes a point of interest, the process flow proceeds to step 137. At step 137, the most recent first still image from the endoscope is marked as a bookmark.

At each pass through step 137, a bookmark is defined to include the most recent first still image. Because at least one pass is made through step 137, at least one bookmark can be defined during the first endoscopic examination such that each bookmark includes a first still image depicting the point of interest. The computing means can be configured to enable definition of a bookmark during the first endoscopic examination, in which the bookmark includes a first still image depicting the point of interest. In one embodiment, the computing means can be configured to define the bookmark based on an input from a human-machine interface input.

Upon definition of each bookmark, a set of schemas is saved on a permanent basis in the computing means. The set of schemas includes all schemas generated after the commencement of the first endoscopic examination if the current bookmark, i.e., the most recent bookmark, is a first bookmark, or all schemas generated after the immediately preceding bookmark if the current bookmark is not the first bookmark. In other words, upon definition of each of at least one bookmark, all schemas satisfying one of the two conditions is saved to the computing means. The first condition is that each of the schemas to be saved is generated after the commencement of the first endoscopic examination and no prior saving of schemas occurred. The second condition is that a prior saving of schemas occurred during the first endoscopic examination and that each of the schemas to be saved is generated after an immediately preceding saving of schemas. In one embodiment, the computing means can be configured to perform a step of saving to a database, upon definition of each bookmark, all schemas generated after an immediately preceding saving of schemas (if any such preceding saving of schemas occurred) or all schemas after beginning of the first endoscopic examination if an immediately preceding saving of schemas does not exist.

At step 138, determination is made as to whether the first endoscopic examination is complete. This decision can be made by the operator of the endoscope based on the operator's analysis of the images generated by the endoscope up to this point and the operator's information about the subject, which can be provided by methods other than the first endoscopic examination.

If the first endoscopic examination is determined to be complete at step 138, the process flow proceeds to step 139, at which the generation of the schemas is complete and all schemas are stored in a data storage device, which may be located in the computing means employed to control the data acquisition through the endoscope during the first endoscopic examination, or may be located outside the computing means employed to control the data acquisition through the endoscope during the first endoscopic examination. A complete set of schemas stored in the data storage device includes at least one set of schemas, in which each set of schema includes either all schemas up to a schema including the first bookmark or all schemas after a bookmark and up to the next bookmark.

If the first endoscopic examination is determined not to be complete at step 138, the process flow proceeds to step 136, at which the distal end of the endoscope moves further to generate at least one additional first still image. The process flow loops through steps 136, 131, 132, 133, 135, and 135 repeatedly until determination is made that the most recent still image includes another point of interest at step 135. The process flow then proceeds to steps 137 and 138. At step 138, another determination is made as to whether the examination is complete, and depending on the result of this determination, the process flow can proceed to step 139 or to the looping steps of 136, 131, 132, 133, 134, and 135. Upon completion of step 139 in the second flow chart in FIG. 3, the step 130 in the first flow chart in FIG. 2 is completed.

Referring to step 150 in the first flow chart of FIG. 2, the first endoscopic examination is ended.

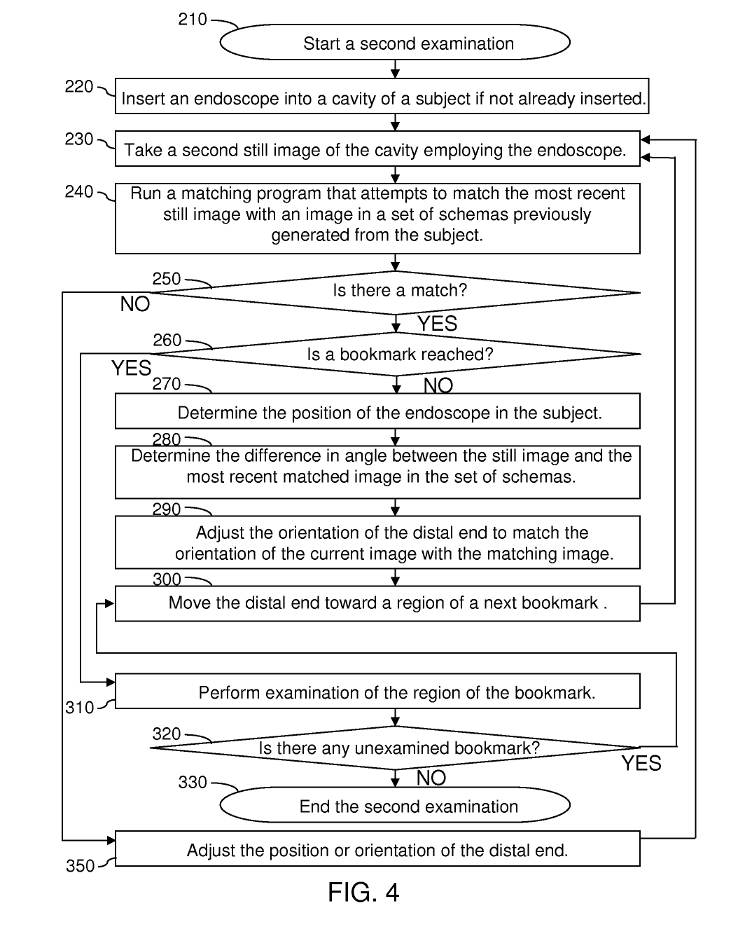

A second endoscopic examination can be performed at any time after the first endoscopic examination in the same cavity of the same subject. The time period between the first endoscopic examination and the second examination depends on the nature of the first and second endoscopic examinations. Referring to FIG. 4, a second flow chart illustrates an exemplary sequence of processing steps for a second endoscopic examination.

Referring to step 210, the second endoscopic examination begins by providing an endoscope and the subject including the cavity that was previously examined during the first examination. The endoscope employed during the second endoscopic examination can be the same as, or different from, the endoscope that was previously employed during the first endoscopic examination.

Referring to step 220, the endoscope employed for the second endoscopic examination is inserted into the cavity of the subject at the same entry point as the entry point employed for the first endoscopic examination. In case the second endoscopic examination immediately follows the first endoscopic examination before the endoscope employed the first endoscopic examination is removed, the insertion of the endoscope may not be necessary and the endoscope employed for the first endoscopic examination can be employed for the second endoscopic examination. If the second endoscopic examination is performed after the endoscope is removed at the end of the first examination, the same endoscope or a different endoscope is inserted into the cavity of the subject.

Referring to step 230, as an endoscope moves through the cavity during the second endoscopic examination, a second still image of a current view is generated by the endoscope. The second still image herein refers to a still image generated during the second endoscopic examination. In one embodiment, the generation of the second still image can be triggered by an operator who provides a manually-generated control signal to the endoscope either through a human-machine interface device (e.g., a button, a lever, etc.) or by remote control. The manually-generated control signal can be directly applied to the endoscope, or can be applied to a computing means for subsequent transmission to the endoscope. The computing means may provide other general control signals to, and/or receives data from, the endoscope. The manually-generated control signal can be generated by the operator as needed to facilitate the navigation of the distal end during the second endoscopic examination.

In another embodiment, the generation of second still images can be triggered at each moment or location of the distal end selected according to a predetermined plan. For example, second still images can be generated at regular time intervals during the movement of the distal end along the cavity. The regular time intervals employed for generation of second still images can be predetermined prior to commencement of the second endoscopic examination, or can be determined or changed during the second endoscopic examination. In yet another embodiment, the generation of the second still images can be conditioned upon a fixed time delay upon commence of movement of the distal end, which can be detected by changes in the current view of the endoscope, changes in a measured inclination of the endoscope, or a combination thereof. In still another embodiment, the generation of the second still images can be conditioned upon any combination of multiple factors including a manually-generated control signal, passage of time, and/or a time delay in combination with detection of movement of the distal end. The second still image generated by the endoscope is stored in the computing means for use in subsequent steps.

Referring to step 240, a matching program is run in the computing means. The matching program attempts to match the most recent second still image with an image in a set of schemas previously generated from the subject during the first examination. To run the matching program, data for the set of schemas is retrieved from a data storage device. This data storage device can be the same data storage device to which the complete set of schemas is stored during the first endoscopic examination or a different data storage device to which the data representing the complete set of schemas is transferred from the data storage device to which the complete set of schemas is stored during the first endoscopic examination. The data storage device from which the set of schemas is retrieved can be a stand-alone data storage device, or can be embedded in the computing means that controls the second endoscopic examination.

As discussed above, each schema includes a first still image of a region in the cavity of the subject and the direction of the distal end of the endoscope employed for the first endoscopic examination. The matching program finds a matching image among the set of first still images in the retrieved set of schemas. The matching image is identified by the matching program when the matching program determines that the matching image and the second still image depict a same region in the cavity. Any image matching algorithm known in the art may be employed to identify the matching image from among the retrieved set of schemas.

The retrieved set of schemas can be the same as the complete set of schemas generated in the first endoscopic examination, or can be a subset of the complete set of schemas that includes less than all schemas in the complete set of schemas. In one embodiment, the matching program compares all first still images in the retrieved set of schemas. In another embodiment, the matching program can enhance the efficiency of image comparison by limiting the first still images to be searched within a limited range. The limitation on the range of the first still images based on the information available to the computing means or based on the information known to the operator.

In one embodiment, search of the set of first still images for the finding of the matching image can limited to at least one first still image saved in the same saving operation of schemas during the first endoscopic examination. In this case, the at least one first still image saved in the same saving operation of schemas during the first endoscopic examination include first still images that are generated prior to, or is the same as, a first still image corresponding to a navigation target bookmark, i.e., a bookmark including a point of interest that the operator intends to navigate the endoscope to, and are generated after definition of any other previous bookmark, i.e., all other bookmarks that precede the definition of the navigation target bookmark. Thus, search of the set of first still images for the finding of the matching image can be limited to at least one first still image generated prior to, or is the same as, a first still image corresponding to the bookmark and generated after definition of any other bookmark that precedes definition of the bookmark.

Referring to step 250, determination is made as to whether a matching image is successfully identified by the matching program. The determination can be made by the image matching program running on the computing means. A manual override on the decision can be provided by the operator of the endoscope. If the matching program fails to identify a matching image, the process flow proceeds from step 250 to step 350. At step 350, the position and/or orientation of the distal end can be adjusted based on the operator's discretion. Alternately, the position and/or orientation of the distal end can be adjusted based on a predetermined algorithm executed by a program running in the computing means and prompts the operator with a recommended type of adjustment of the distal end. Upon adjustment of the position and/or the orientation of the endoscope, the process flow proceeds to step 230 to take another second still image.

If the matching program successfully identifies a matching image, the process flow proceeds from step 250 to step 260. At step 260, determination is made as to whether a bookmark is reached. A bookmark includes a point of interest previously defined during the first endoscopic examination. Therefore, the matching image is a bookmark if the matching image depicts a point of interest, and the matching image is not a bookmark if the matching image does not depict a point of interest.

If the matching image depicts a point of interest, the distal end is at a location corresponding to the bookmark. The computing means can then prompt the operator of the endoscope for examination of the point of interest as viewed through the endoscope. The process flow then proceeds to step 310, at which examination of the region of the bookmark, i.e., the region shown in the current view of the endoscope, is performed by the operator of the endoscope. The examination of the region of the bookmark can include observation by the operator, taking of additional images by the operator, and/or a collateral operation on any portion of the region within the current view of the endoscope including, but not limited to, surgical operation on a portion of the region within the current view.

Referring to step 320, determination is made as to whether there is any unexamined bookmark. The determination can be made by the image matching program running on the computing means by comparing a complete list of bookmarks generated in the first endoscopic examination with a list of all bookmarks examined during the second endoscopic examination up to that time. A manual override on the decision can be provided by the operator of the endoscope. If all bookmarks generated in the first endoscopic examination has been examined and there is no unexamined bookmark, the process flow proceeds from step 320 to step 330. At step 330, the second endoscopic examination is terminated.

If there is any unexamined bookmark left among the complete set of bookmarks generated in the first endoscopic examination, the process flow proceeds to step 330 to move the distal end toward a region of a next bookmark. The process flow then proceeds to step 230 to take another second still image.

Referring back to step 260, the matching image does not depicts a point of interest, the distal end is at a location that does not correspond to a bookmark. The process flow then proceeds to step 270, at which information for further navigation of the distal end is generated by analyzing the matching image and the most recent second still image.

Specifically, at step 270, the computing means determines the position of the distal end based on comparison of the matching image and the most recent second still image. Specifically, the position of the distal end is determined relative to the regions captured in each of the first still images. Particularly, the position of the distal end is determined relative to the regions in the cavity depicted in the matching image and additional first still images immediately before and/or after the matching image. Further, the position of the distal end can be determined relative to the region corresponding to the next bookmark that the operator intends to navigate the distal end to. If the first still images are generated at regular time intervals, it is possible to estimate a navigation time from the current position of the distal end to the region corresponding to the next bookmark.

Referring to step 280, the computing means determines the relative angle α between the matching image and the most recent second still image based on comparison of the matching image and the most recent second still image. The relative angle α can be determined by comparing orientations of the features common to the matching image and the most recent second still image.

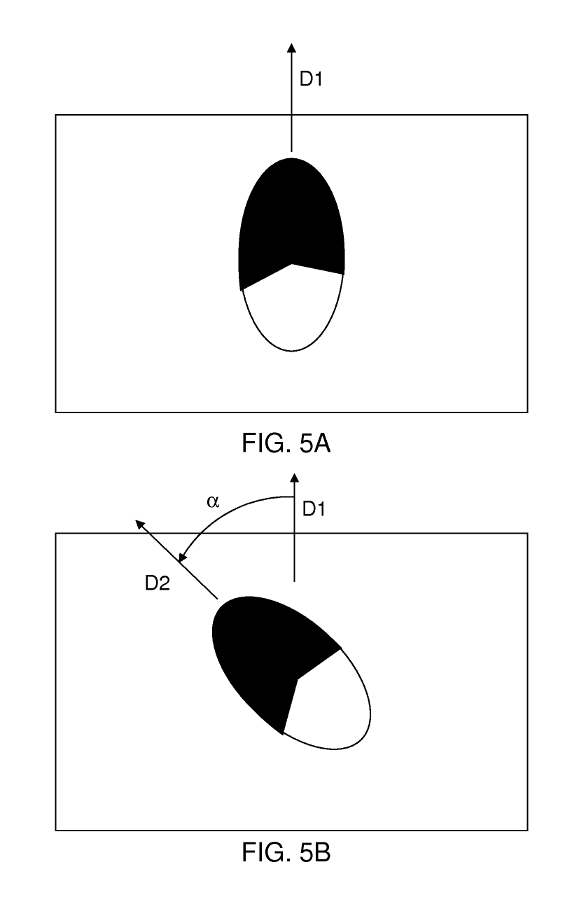

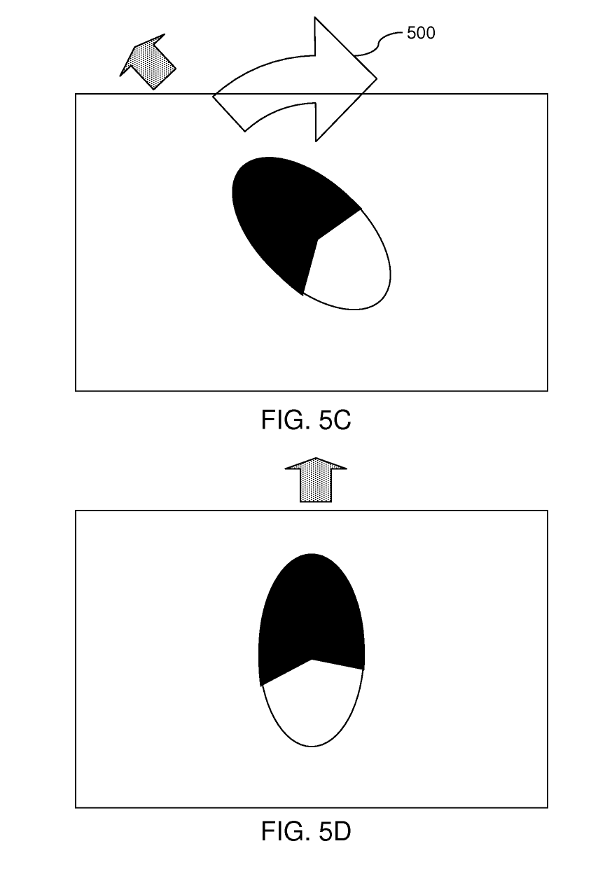

FIGS. 5A and 5B schematically illustrate the determination of the relative angle α between the matching image and the most recent second still image. FIG. 5A schematically illustrates an exemplary matching image, which is a first exemplary still image generated during the first endoscopic examination and is determined to depict the same region as the most recent second still image generated during the second endoscopic examination. FIG. 5B schematically illustrates a second exemplary still image, which is the most recent second still image generated during the second endoscopic examination.

The image matching program executed in the computing means identifies at least one common feature among the matching image and the most recent second still image that forms the basis of determining that the identified first still image is the matching image. The direction of the distal end employed for the first endoscopic examination is identified in the matching image as a first direction D1, which is shown by an arrow labeled “D1” in FIG. 5A. The direction of the distal end in the most recent second still image is identified as a second direction D2, which is shown by an arrow labeled “D2” in FIG. 5B. The relative angle α is the angle between the first direction D1 and the second direction D2, and can be computed by the image analysis program executed in the computing means. In some embodiments, the direction of the distal end can be identified by an up/down movement of the distal end immediately before or immediately after taking a still image. In some other embodiments, the direction of the distal end can be identified by a marker pixel or the orientation of the still image. Any other method of identifying the direction of the distal end may be employed at the time of generation of the first and second still images.

While the third flow chart illustrates an embodiment in which step 270 is performed before step 280, other embodiments can also be implemented in which step 280 is performed before, or simultaneously with, step 270.

At steps 290 and 300, the distal end is subsequently navigated employing the information generated in steps 270 and 280. Specifically, the distal end can be navigated by linearly moving the distal end or by rotating the distal end based on information on the determined position or the determined relative angle α.

Referring to step 290, the orientation of the distal end can be adjusted to match the orientation of the current image from the endoscope with the orientation of the distal end in the matching image. FIGS. 5C and 5D schematically illustrate the matching of the orientation of the current view with the orientation of the distal end in the matching image.

Referring to FIG. 5C, upon determination of the relative angle α, a cursor can be displayed on a display means in a direction corresponding to the current direction of the distal end. The cursor can be any type of prompt displayed on the display means, and can be graphic or alphanumeric. For example, the cursor can be in the shape of an arrow displayed on the display means. At this point, the current image is substantially identical with the most recent second still image. As such, the cursor can point along an orientation of the second exemplary still image that corresponds to the current orientation of the distal end, which is substantially the same as the orientation of the distal end during generation of the most recent second still image.

In one embodiment, the computing means can be configured to prompt rotation of the distal end based on information on the determined relative angle α. In this embodiment, the computing means can generate an instruction prompt 500, which provides an instruction to the operator to make a rotational adjustment to the distal end. The instruction prompt 500 can be a graphic prompt or a text prompt. As illustrated in FIG. 5D, the operator can then adjust the rotation of the distal end until the orientation of the distal end in the current view matches the orientation of the distal end as determined in the matching image.

In another embodiment, the computing means can be configured to automatically navigate the distal end by rotating the distal end based on information on the determined position or the determined relative angle α until the rotation of the distal end until the orientation of the distal end in the current view matches the orientation of the distal end as determined in the matching image as illustrate in FIG. 5D.

Referring to step 300, the distal end is linearly moved toward a region of the next bookmark, which includes the next point of interest to be examined. The amount of linear advance (or retreat) during the linear movement can be determined based on the position of the distal end as determined relative to the next region of interest as determined at step 270.

While the third flow chart illustrates an embodiment in which step 290 is performed before step 300, other embodiments can also be implemented in which step 300 is performed before, or simultaneously with, step 290. In the combined steps of 290 and 300, the distal end is navigated by linearly moving the distal end and by rotating the distal end based on information on the determined position and the determined relative angle α.

In one embodiment, the computing means can be configured to prompt navigation of the distal end based on information on the determined position or the determined relative angle α. In this embodiment, the operator makes a final decision as to the degree of linear movement and the angular rotation of the distal end. In an alternate embodiment, the computing means can be configured to automatically navigate the distal end by linearly moving the distal end and/or by rotating the distal end based on information on the determined position and/or the determined relative angle α.

After moving the endoscope, the process flow proceeds to step 230 to take additional second image at a new position of the distal end. Steps 230, 240, 250, 260, 270, and 280 can be repetitively performed until the distal end reaches a point of interest, which is determined at step 260. Thus, the computing means can be configured to perform the steps of:

(a) generating an additional second still image in the second endoscopic examination of the cavity (corresponding to additional passes through step 230);

(b) finding an additional matching image among the set of first still images, wherein the additional matching image and the additional second still image include a same region in the cavity (corresponding to additional passes through step 240 and 250); and

(c) determining an additional position of the distal end and an additional relative angle between the additional matching image and the additional second still image based on comparison of the additional matching image and the additional second still image (corresponding to steps 270 and 280).

In each loop of steps in which steps 270 and 280 are performed, steps 290 and 300 are performed after steps 270 and 280 so that the distal end is navigated by linearly moving the distal end and/or by rotating the distal end based on information on the determined additional position and/or the determined additional relative angle. Steps 290 and 300 can be implemented in various embodiments. In one embodiment, the computing means can be configured to perform, until the distal end reaches a point of interest, the step(s) of prompting navigation of the distal end based on information on the determined additional position or the determined additional relative angle after each step of determining the additional position and the additional relative angle. In another embodiment, the computing means can be configured to perform, until the distal end reaches a point of interest, the step(s) of automatically navigating the distal end based on information on the determined additional position or the determined additional relative angle after each step of determining the additional position and the additional relative angle.

By employing the methods of the third flow chart, the endoscope employed for the second endoscopic examination follows the path of the endoscope employed for the first endoscopic examination. Further, the endoscope employed for the second endoscopic examination can reach and examine each point of interest identified as a bookmark during the first endoscopic examination. Thus, the efficiency of navigation during the second endoscopic examination is enhanced by utilizing the graphic data generated during the first endoscopic examination in the form of a set of schemas.

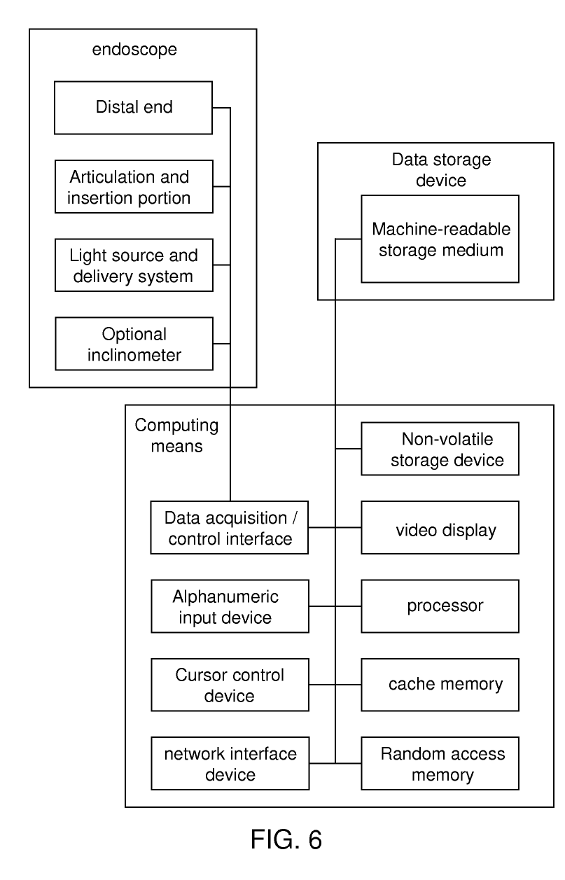

Referring to FIG. 6, an exemplary system configured to perform endoscopic examinations according to an embodiment of the present disclosure is illustrated. The exemplary system includes an endoscope, a computing means, and a data storage device. While the data storage device is shown as a separate unit in the exemplary system, embodiments in which the data storage device is embedded in the computing means can also be implemented.

The endoscope can be any endoscope known in the art provided that the endoscope is configured to electronically transmit data (including still images) to the computing means and to electronically receive instructions from the computing means. The electronic communication between the endoscope and the computing means can be effected through signal cables or by wireless communication. The computing means can be a stand-alone computer such as a general purpose personal computer (PC) configured to run a program controlling the endoscope along with other programs or a dedicated special purpose computer configured to run only a program controlling the endoscope. Alternately, the computing means can be a computing device embedded in the operating portion or embedded in a different instrument. The data storage device can be any device configured to store electronic data on a permanent basis, and can be a stand-alone data storage unit or a server connected to the computing means, or can be incorporated into the computing means. Alternately, the data storage device can be a portable data storage device that can interface the computing unit through any known type of interface including a universal serial bus (USB).

The endoscope can include, for example, a distal end, an articlating section, an insertion portion including optical fibers, a light source and delivery system, and optionally, an inclinometer attached to, or incorporated into, the distal end. The distal end includes an illumination device connected to the light delivery system as well as at least one of optics system and a camera as known in the art so that an image can be transmitted through the insertion portion to the operating portion and eventually to the computing means. The computing means can include a processor, a cache memory, a random access memory, a video display, a non-volatile storage device such as a hard drive, a data acquisition/control interface device that receives data and transmits instructions to the endoscope, an alphanumeric input device such as a keyboard, a cursor control device such as a mouse or a track ball, and a network interface device. The processor, the cache memory, and the random access memory collectively form a processing unit of the computing means. The data storage device includes a machine-readable storage medium, which can be a hard disk or a portable data storage unit. The components of each of the endoscope, the computing means, and the data storage device are illustrative and non-limiting examples. As long as the functionality of each of the endoscope, the computing means, and the data storage device can be provided, any of the components illustrated in FIG. 6 can be replaced with an equivalent functional component. Further, some components can be added or transferred from one unit to another unit. For example, an additional video display can be added to the endoscope and/or the video display can be transferred from the computing means to the endoscope.

The various steps in the first, second, and third flow charts can be performed by the computing means employing an automated program. The computing means can be configured to store the program and/or the data employed to perform the methods of the present disclosure in a non-transitory machine-readable data storage device. The information stored in the non-transitory machine-readable data storage device can be transmitted to the endoscope by signal transmission through wired communication, wireless communication, or transport of a non-transitory machine-readable data storage device if the non-transitory machine-readable data storage device is portable. Further, the computing means can be configured to interface with multiple or alternate endoscopes so that different endoscope can be employed during the first endoscopic examination and the second endoscopic examination. The computing means can be configured to perform both the first and second endoscopic examinations employing the same endoscope or different endoscopes. Further, the methods of the present disclosure can be performed employing two separate systems such that a first system includes a first endoscope, a first computing means, and a first data storage device and a second system includes a second endoscope, a second computing system, and a second data storage device provided that data can be transferred between the first data storage device and the second data storage device. In a variation of this embodiment, a common data storage device can be employed in lieu of the first data storage device and the second data storage device.

Further, a non-transitory machine-readable data storage device can be employed to embody a program of machine-readable instructions that can be performed in the computing means. The machine-readable instructions can include steps in the various flow charts in FIGS. 2, 3, 4, or any portion thereof, or any combination thereof. The program of machine-readable instructions can be transferred to the computing means to perform the various steps described above.

The program for performing the various steps in the first, second, and third flow charts can be stored in a data storage device. The data storage device can be a non-volatile storage device embedded in the computing means in FIG. 6, the data storage device illustrated in FIG. 6, or a portable data storage device (not shown). The data storage device is programmable and readable by a machine and tangibly embodies or stores a program of machine-executable instructions that are executable by the machine to perform the methods described herein are also provided. For example, the automated program can be embodied, i.e., stored, in a machine-readable data storage devices such as a hard disk, a CD ROM, a DVD ROM, a portable storage device having an interface such as a USB interface, a magnetic disk, or any other storage medium suitable for storing digital data.

The computer program product can comprise all the respective features enabling the implementation of the inventive method described herein, and which is able to carry out the method when loaded in a computer system. Computer program, software program, program, or software, in the present context means any expression, in any language, code or notation, of a set of instructions intended to cause a system having an information processing capability to perform a particular function either directly or after either or both of the following: (a) conversion to another language, code or notation; and/or (b) reproduction in a different material form. The computer program product can be stored on hard disk drives within a processing unit in the computing means or can be located on a remote system such as a server (not shown), coupled to the processing unit in the computing means, via a network interface such as an Ethernet interface.

While the disclosure has been described in terms of specific embodiments, it is evident in view of the foregoing description that numerous alternatives, modifications and variations will be apparent to those skilled in the art. Accordingly, the disclosure is intended to encompass all such alternatives, modifications and variations which fall within the scope and spirit of the disclosure and the following claims.

What is claimed is:

1. A system for endoscopic examination comprising an endoscope and a computing means, wherein said computing means is configured to perform the steps of:
storing data for a set of schemas generated during a first endoscopic examination employing a first endoscope having a first distal end, each schema including a first still image of a region in a cavity of a subject and a direction of said first distal end at a time of taking said first still image, wherein said first endoscope is said endoscope or another endoscope;
storing a second still image generated by said endoscope during a second endoscopic examination of said cavity; and
finding a matching image among a set of first still images in said set of schemas, wherein said matching image and said second still image depict a same region in said cavity.

2. The system of claim 1, wherein said computing means is configured to perform a step of determining a position of a distal end of said endoscope and a relative angle between said matching image and said second still image based on comparison of said matching image and said second still image.

3. The system of claim 2, wherein said computing means is configured to perform a step of prompting navigation of said distal end based on information on said determined position or said determined relative angle.

4. The system of claim 2, wherein said computing means is configured to perform a step of automatically navigating said distal end by linearly moving said distal end or by rotating said distal end based on information on said determined position or said determined relative angle.

5. The system of claim 2, wherein said computing means is configured to repetitively perform, until said distal end reaches a point of interest, steps of:
generating an additional second still image in said second endoscopic examination of said cavity;
finding an additional matching image among said set of first still images, wherein said additional matching image and said additional second still image include a same region in said cavity; and
determining an additional position of said distal end and an additional relative angle between said additional matching image and said additional second still image based on comparison of said additional matching image and said additional second still image.

6. The system of claim 5, wherein said computing means is configured to perform, until said distal end reaches a point of interest, a step of prompting navigation of said distal end based on information on said determined additional position or said determined additional relative angle after each step of determining said additional position and said additional relative angle.

7. The system of claim 5, wherein said computing means is configured to perform, until said distal end reaches a point of interest, a step of automatically navigating said distal end based on information on said determined additional position or said determined additional relative angle after each step of determining said additional position and said additional relative angle.

8. The system of claim 1, wherein said endoscope and said computing means configured to enable generation of a set of first still images in said first endoscopic examination, and said system is configured to generate said set of schemas employing said set of first still images.

9. The system of claim 8, wherein said computing means is configured to enable definition of a bookmark during said first endoscopic examination, wherein said bookmark includes a first still image depicting said point of interest.

10. The system of claim 9, wherein said computing means is configured to define said bookmark based on an input from a human-machine interface input.

11. The system of claim 9, wherein said computing means is configured to perform steps of:
determining whether said matching image depicts said point of interest; and
prompting examination of, if said matching image depicts said point of interest, said point of interest as viewed through said endoscope.

12. The system of claim 9, wherein said computing means is configured to perform a step of saving to a database, upon definition of said bookmark, all schemas generated after an immediately preceding saving of schemas or all schemas after beginning of said first endoscopic examination if an immediately preceding saving of schemas does not exist.

13. The system of claim 12, wherein said computing means is configured to perform a step of retrieving said set of schemas from said database for said finding of said matching image, wherein search of said set of first still images for said finding of said matching image is limited to at least one first still image generated prior to, or is the same as, a first still image corresponding to said bookmark and generated after definition of any other bookmark that precedes definition of said bookmark.

14. A method of operating at least one endoscope comprising:
generating a set of schemas in a first endoscopic examination of a cavity of a subject employing a first endoscope having a first distal end, each schema including a first still image of a region in a cavity of a subject and a direction of said first distal end at a time of taking said first still image;
generating a second still image in a second endoscopic examination of said cavity employing an endoscope having a distal end, wherein said endoscope is said first endoscope or another endoscope; and
finding a matching image among a set of first still images in said set of schemas, wherein said matching image and said second still image depict a same region in said cavity.

15. The method of claim 14, further comprising:
determining a position of said distal end and a relative angle between said matching image and said second still image based on comparison of said matching image and said second still image; and
navigating said distal end by linearly moving said distal end or by rotating said distal end based on information on said determined position or said determined relative angle.

16. The method of claim 15, further comprising repetitively performing, until said distal end reaches a point of interest, steps of:
generating an additional second still image in said second endoscopic examination of said cavity;
finding an additional matching image among said set of first still images, wherein said additional matching image and said additional second still image include a same region in said cavity;
determining an additional position of said distal end and an additional relative angle between said additional matching image and said additional second still image based on comparison of said additional matching image and said additional second still image; and
navigating said distal end by linearly moving said distal end or by rotating said distal end based on information on said determined additional position or said determined additional relative angle.

17. The method of claim 16, further comprising defining a bookmark during said first endoscopic examination, wherein said bookmark includes a first still image depicting said point of interest.

18. The method of claim 14, further comprising defining at least one first still image as at least one bookmark during said first endoscopic examination, wherein each of said at least one bookmark depicts a point of interest.

19. The method of claim 18, further comprising:
determining whether said matching image depicts one of at least one point of interest in said at least one bookmark; and
examining, if said matching image depicts one of at least one point of interest in said at least one bookmark, a point of interest as viewed through an endoscope.

20. The method of claim 19, further comprising:
determining a position of said distal end and a relative angle between said matching image and said second still image based on comparison of said matching image and said second still image; and
navigating said distal end by rotating said distal end based on information on said determined relative angle and by linearly moving said distal end toward a point of interest in one of said at least one bookmark based on said determined position.

21. The method of claim 18, further comprising saving to a database, upon definition of each of said at least one bookmark, all schemas generated after an immediately preceding saving of schemas or all schemas after beginning of said first endoscopic examination if an immediately preceding saving of schemas does not exist.

22. The method of claim 21, further comprising retrieving said set of schemas from said database for said finding of said matching image, wherein search of said set of first still images for said finding of said matching image is limited to at least one first still image generated prior to, or is the same as, a first still image corresponding to said one of said at least one bookmark and generated after definition of any other bookmark that precedes definition of said at least one bookmark.

23. A non-transitory machine-readable data storage device embodying a program of machine-readable instructions that can be performed in a computing means, wherein said machine-readable instructions includes steps for:
storing data for a set of schemas generated during a first endoscopic examination employing a first endoscope having a first distal end, each schema including a first still image of a region in a cavity of a subject and a direction of said first distal end at a time of taking said first still image;
storing a second still image generated by an endoscope having a distal end during a second endoscopic examination of said cavity, wherein said endoscope is said first endoscope or another endoscope; and
finding a matching image among a set of first still images in said set of schemas, wherein said matching image and said second still image depict a same region in said cavity

24. The non-transitory machine-readable data storage device of claim 23, wherein said machine-readable instructions includes steps for determining a position of said distal end and a relative angle between said matching image and said second still image based on comparison of said matching image and said second still image.

25. The non-transitory machine-readable data storage device of claim 24, wherein said machine-readable instructions includes steps for prompting navigation of said distal end toward a point of interest defined during said first endoscopic examination based on information on said determined position or said determined relative angle.
